(19) United States
(12) Patent Application Publication
(10) Pub. No.: US 2023/0211072 A1
(43) Pub. Date: Jul. 6, 2023

(54) SYSTEMS AND METHODS FOR PROGRAMMING A MEDICAL INFUSION PUMP

(71) Applicant: Smiths Medical ASD, Inc., Plymouth, MN (US)

(72) Inventors: Kurt L. ELLIASON, Maple Grove, MN (US); Timothy E. EALY, New Prague, MN (US)

(21) Appl. No.: 17/927,805

(22) PCT Filed: May 26, 2021
(86) PCT No.: PCT/US2021/070605

§ 371 Date: Nov. 25, 2022

Related U.S. Application Data
(60) Provisional application No. 63/030,724, filed on May 27, 2020.

(51) Int. Cl. A61M 5/142; A61M 5/172; G16H 20/17; G16H 40/40

(52) U.S. Cl. CPC A61M 5/142; A61M 5/172; G16H 20/17; G16H 40/40; A61M 2205/50

(57) ABSTRACT

Systems and methods for programming an infusion pump. Parameters provided in an infusion order from a Hospital Information System (HIS) are used by the infusion pump to find a matching entry in the infusion pump drug library. If there is no matching entry in the infusion pump drug library, then a manual mode is populated with the parameters and the pump can accordingly be operated in the manual mode.

### CROSS-REFERENCE TO RELATED APPLICATIONS

The present application claims priority to U.S. Provisional Application No. 63/030,724, filed on May 27, 2020, which is hereby fully incorporated herein by reference.

### TECHNICAL FIELD

Embodiments relate generally to medical devices and, more particularly, to programming infusion pumps.

### BACKGROUND

In the medical arts, infusion pumps have been useful for managing the delivery and dispensation of a prescribed amount or dose of a drug, fluid, fluid-like substance, or medicament (hereinafter, collectively, an “infusate” or “infusates”) to patients. Infusion pumps can provide some significant advantages over manual infusion techniques, by accurately delivering and dispensing infusates over an extended period of time.

Infusion pumps are particularly useful for treating diseases and disorders that require regular pharmacological intervention, including cancer, diabetes, and vascular, neurological, and metabolic disorders. They also enhance the ability of healthcare providers to deliver anesthesia and manage pain. Infusion pumps are used in various settings, including hospitals, nursing homes, and other short-term and long-term medical facilities, as well as in residential care settings. Infusion pumps can include various constructions, modes of operation, and types.

Generally, infusion pumps can comprise a variety of types of pumps. In some cases, infusion pumps include syringe pumps, large volume pumps (“LVP”), and patient-controlled analgesia (“PCA”) pumps. Depending upon their specific designs and intended uses, infusion pumps can be used to administer infusates through various delivery methods, including intravenously, intraperitoneally, intra-arterially, subcutaneously, neuraxially, and specifically into an intraoperative site, epidural space, and subarachnoid space.

Infusion pumps may be controlled locally via the programming of each individual pump. For example, a medical practitioner can configure an infusion pump to execute a delivery profile that corresponds to a patient's treatment needs, or a patient can configure an infusion pump according to their individual requirements within pre-defined limits without the involvement of a physician. Alternatively, infusion pumps may be controlled via other techniques such as by, for example, a network server that communicates with the pumps. Hospital information systems (HIS) and electronic medical record (EMR) systems may, in some circumstances, provide such functionality by provision of, for example, drug libraries, auto-charting, and other “smart” communication systems that operate in coordination with the infusion pumps.

Also, some infusion pump systems include a “Bar-Coded Medication Administration” (“BCMA”) process that may communicate with HIS and/or EMR systems to reduce medication delivery errors and enhance patient safety. In a typical BCMA process, individual infusate containers such as syringes (for use with, e.g., syringe pumps) and bags (for use with, e.g., LVPs) are labeled with unique barcodes that correspond to a particular patient. If the BCMA system cannot match the infusate with an applicable order for a patient in the HIS, EMR, or drug library, a warning is communicated to the practitioner. In some infusion systems, the BCMA process is a first step in starting operation of an infusion pump.

Currently, such interoperability between HIS and EMR systems with infusion pumps is somewhat limited. Although interoperability generally increases patient safety and operational efficiency, it is expensive and challenging to implement. In particular, it is difficult to ensure that an HIS/EMR system aligns and is kept in sync with each pump (e.g. the drug library) to implement an autoprogramming or smart pump programming (“SPP”) workflow. Further, if an autoprogramming order does not match a pump's drug library, the autoprogramming workflow is exited. In order to implement that order, a new drug library entry must be created or filled in from an empty drug library entry template.

Therefore, there is a need for flexibility in the autoprogramming or smart pump programming workflow.

### SUMMARY

Embodiments described herein substantially meet the aforementioned needs of infusion pump systems. Systems and methods described herein provide usable, flexible workflows for programming an infusion pump with an HIS/EMR. By adding flexibility to an autoprogramming workflow, patient safety is increased. Further, the pump systems can be operated more efficiently by the user.

In general, an autoprogramming workflow typically begins with a computerized physician order entry (CPOE) for an infusion by a physician user at a Hospital Informaton System (HIS) or Electronic Medical Records (EMR) system (hereinafter, collectively, “HIS”). An infusion order can be electronically communicated to an operably coupled pharmacy subsystem, where the infusion order is verified. The infusion order can then be electronically communicated to an operably coupled infusion pump where the infusion order can be approved and started. In embodiments, the infusion can be started automatically. But for safety purposes, an attending healthcare professional typically approves or starts the infusion at the pump with minimal interaction with the pump. This workflow eliminates manual entry errors at the pump and is typically more streamlined and efficient than manual entry of infusion parameters.

However, to program an infusion pump using the autoprogramming workflow, the infusion order must have a corresponding matching entry in the drug library for a particular pump. Keeping all drug libraries for all pumps updated and matching the HIS system can be difficult. And, traditionally, without a match of the infusion order in the drug library at the pump, the pump exits the autoprogramming workflow.

In a feature and advantage of embodiments, flexibility is built into an autoprogramming workflow. In particular, embodiments of control logic at the pump can intelligently check for a match in the drug library. And, when no match is found, embodiments allow for “manual mode” operation with parameters received from the HIS, by automatically populating the received parameters in the “manual mode.” “Manual mode” operation with the received parameters can be allowed because the parameters have already been verified by a pharmacy subsystem or corresponding safety limit check by or through the HIS. Moreover, an (at least) partially populated library entry does not need to be created on the pump (as is typical in a manual mode operation without an existing library entry).

More particularly, systems and methods use the parameters provided in the infusion order to either find a matching drug library entry or program the pump using a “manual mode” the same way that the clinician would perform such tasks. This allows the flexibility and efficiency of running the pump in a “manual mode” while still having the safety of parameters being populated from the HIS where they have been reviewed and approved. Chances of a mistake made during the parameter entry are thereby greatly reduced.

One particular scenario illustrating this benefit is a situation where a pediatric drug library is set up for 8-year-olds weighing 60 lbs. but the patient is a 16-year-old weighing 160 lbs. This would traditionally cause the practitioner to bypass the HIS pump programming workflow to manually program the pump and then manually chart the infusion. However, in embodiments described by example herein, by staying within the HIS pump programming workflow, the programming of the pump and charting is still automatic using the already-approved parameters. Another scenario illustrating the benefit would be a drug shortage that necessitates a substitution of one drug for another that is not specifically listed in the library.

Accordingly, staying within the autoprogramming workflow in this hybrid manner relieves the practitioner from having to enter a significant number of keystrokes as in typical manual entry. Removing keystrokes thus decreases the potential for programming errors and increases patient safety.

In another feature and advantage of embodiments, by staying within the autoprogramming workflow, data about the infusion can automatically flow back to the HIS for recording in the system. Traditionally, in a manual programming mode, data about an infusion would need to be manually entered in the system. Accordingly, charting is thereby better automated.

In another feature and advantage of embodiments, drug library maintenance is improved. In particular, when an infusion is not matched, the data about a resulting “no-match” indication, as well as a further manual override infusion that was actually programmed is automatically queued and sent back to the HIS or pharmacy subsystem. Therefore, an association is created between the no-match and the actual infusion subsequently programmed (compared to traditional systems in which the further actual infusion is usually not connected to the no-match indication).

Consequently, when looking at changes to pump drug libraries, a user such as a pharmacist has actionable data of how a particular infusion was attempted, that the drug library did not have a corresponding match, and the infusion protocol that a clinician then programmed. Appropriate updates to the pump drug libraries and/or autoprogramming workflow can then be made.

In an embodiment, an infusion pump includes a processor, memory operably coupled to the processor, and control logic comprising instructions that, when executed, cause the processor to: receive programming parameters corresponding to an infusion order from an HIS, the programming parameters having been verified by the HIS; determine if the received programming parameters match a drug library protocol in a drug library of the infusion pump; when the programming parameters do not match any drug library protocol in the drug library, queue a no-match error code; populate a manual mode with the received programming parameters; and operate the infusion pump in the manual mode using the populated programming parameters.

In an embodiment, a method of programming an infusion pump comprises receiving programming parameters corresponding to an infusion order from an HIS, the programming parameters having been verified by the HIS; determining if the received programming parameters match a drug library protocol in a drug library of the infusion pump; when the programming parameters do not match any drug library protocol in the drug library, queuing a no-match error code; populating a manual mode with the received programming parameters; and operating the infusion pump in the manual mode using the populated programming parameters.

In an embodiment, an infusion pump comprises means for receiving programming parameters corresponding to an infusion order from an HIS, the programming parameters having been verified by the HIS; means for determining if the received programming parameters match a drug library protocol in a drug library of the infusion pump; when the programming parameters do not match any drug library protocol in the drug library, means for queuing a no-match error code; means for populating a manual mode with the received programming parameters; and means for operating the infusion pump in the manual mode using the populated programming parameters.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is a front corner perspective view of an infusion pump comprising a syringe pump, according to an embodiment.

FIG. 2 is a front view of an infusion pump comprising a syringe pump, according to an embodiment.

FIG. 3 is a system diagram of an infusion pump comprising a syringe pump, according to an embodiment.

FIG. 4 is a block diagram for a controller subsystem for an infusion pump, according to an embodiment.

FIG. 5 is an architecture block diagram for an infusion pump system, according to an embodiment.

FIG. 6 is a flowchart of a method of programming an infusion pump, according to an embodiment.

FIGS. 7A-7B are a flowchart of a method of programming an infusion pump, according to an embodiment.

FIG. 8 is a flowchart of a method of intelligent interoperability between an HIS and an infusion pump, according to an embodiment.

While various embodiments are amenable to various modifications and alternative forms, specifics thereof have been shown by way of example in the drawings and will be described in detail. It should be understood, however, that the intention is not to be limited to the particular embodiments described. On the contrary, the intention is to cover all modifications, equivalents, and alternatives falling within the spirit and scope of the subject matter as defined by the claims.

### DETAILED DESCRIPTION OF THE DRAWINGS

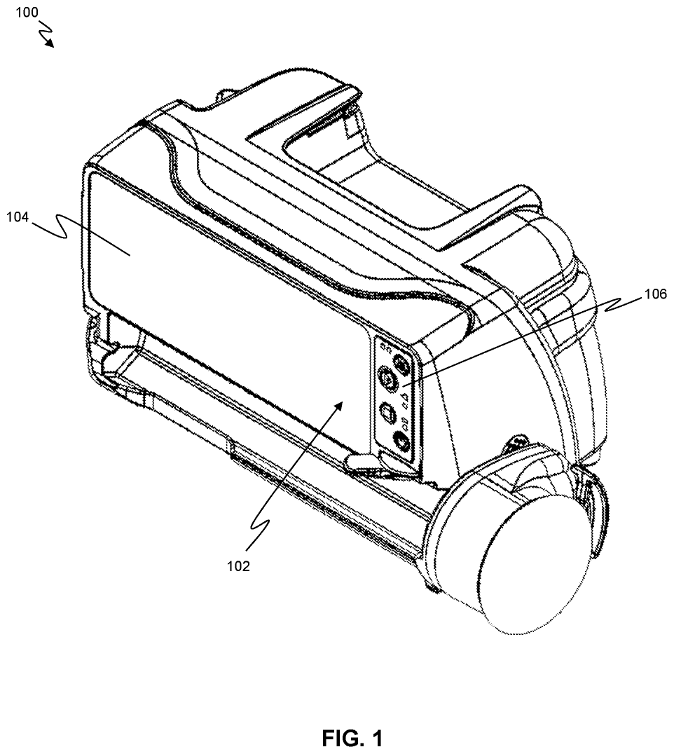

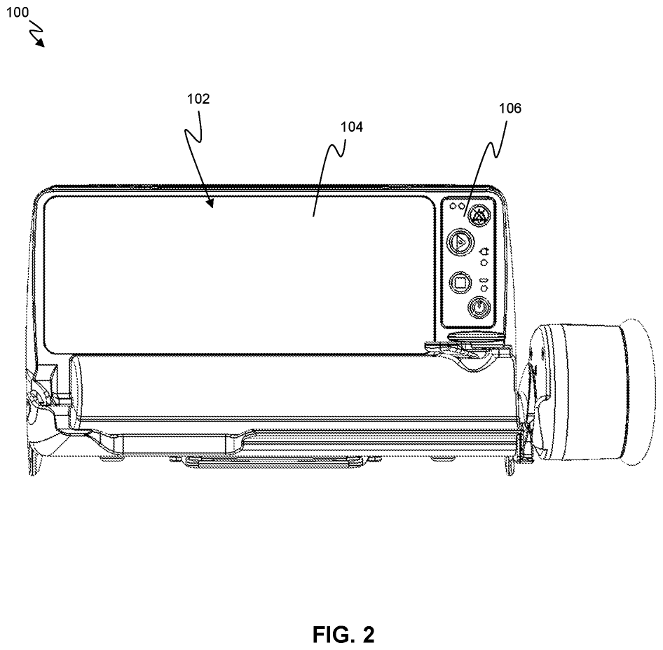

Referring to FIGS. 1-2, an infusion pump, and particularly, a syringe pump 100 is shown. One skilled in the art will readily appreciate that the user experience embodiments described herein can be configured to be utilized with, for example, syringe pumps, large volume pumps, and patient-controlled analgesia (“PCA”) pumps.

“Syringe pumps” generally include pumps for acting on pre-filled medication syringes that are mechanically driven under microprocessor control to deliver prescribed amounts or doses of infusates at controlled rates to patients through infusion lines fluidly connected to the syringes. A syringe pump typically includes a motor that rotates a leadscrew or adjustment mechanism, for example. The leadscrew or adjustment mechanism, in turn, activates a plunger driver of a plunger head which forwardly pushes a plunger within a barrel of the syringe. Pushing the plunger forward then forces the dose of infusate outwardly from the syringe, into the infusion line, and to the patient. As used throughout this disclosure, the term “syringe pump” is intended to generally pertain to any device which acts on a syringe to controllably force fluid outwardly therefrom.

“LVPs” can take on various forms, but are typically infusion pumps coupled to one or more reservoirs configured to hold or store a large amount of medication or fluid to be infused, such as a cassette, IV bag, or other self-contained fluid source. As used throughout this disclosure, the term “LVP” is intended to generally pertain to any infusion pump or device capable of large volume infusion to a patient.

“PCA” pumps can include ambulatory pumps designed to be portable or wearable. In an embodiment, PCA pumps can include SMITHS MEDICAL CADD ambulatory infusion pumps. In embodiments, CADD ambulatory infusion pumps transition easily from hospital care to home care environments.

Pump 100 generally comprises a user interface 102. User interface 102 generally includes a display screen 104 and a keypad 106.

Display screen 104 can be a rectangular, color, LCD screen, and can be a touchscreen in certain embodiments. Display screen 104 can be any type of suitable graphical user interface (“GUI”) for use in controlling pump 100. In some embodiments, display screen 104 can be configured to permit display of four lines of text, up to thirty characters long each. Accordingly, this size of display screen 104 enables viewing information and drug names of significant length. In some embodiments, certain commands or instructions are not controlled by a touchscreen such as display screen 104 but instead are controlled by keypad 106.

Keypad 106 is located adjacent to display screen 104 and presents a variety of buttons and indicator lights. In some embodiments, push buttons requiring physical mechanical actuation are used on keypad 106 for certain user commands, including: on/off power; audible alarm mute; start infusion; and stop infusion. Additional or fewer buttons on keypad 106 are contemplated as well. Physical mechanical actuation buttons, for primary or redundant purposes, provide increased safety and reliability to operators in cases where the touchscreen of display 104 does not function properly, or is otherwise difficult to manipulate correctly. Having a user interface 102 including both a display screen 104 and a keypad 106, accordingly, provides the flexibility of a screen interface as well as the enhanced safety and reliability of physical control buttons. In embodiments, keypad 106 comprises control buttons that can operate functionally in tandem with display screen 104.

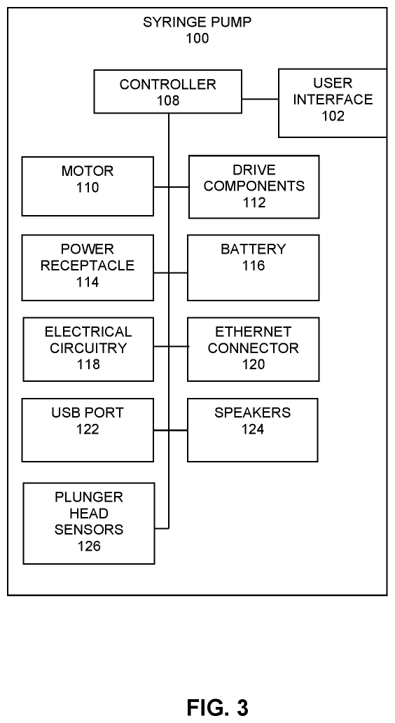

Referring to FIG. 3, a system diagram of pump 100 is depicted, according to an embodiment. FIG. 3 illustrates a syringe pump 100 including user interface 102, a controller 108, a motor 110, drive components (drivetrain) 112, a power receptacle 114, a battery 116, electrical circuitry 118, an Ethernet connector 120, a USB port 122, speakers 124, and plunger head sensors 126.

As will be described, pump 100 and/or its components or subsystems can include a processor or multiple processors. In an embodiment, the processor(s) discussed herein can be any programmable device that accepts digital data as input, is configured to process the input according to instructions or algorithms, and provides results as outputs. In an embodiment, the processor(s) discussed herein can be configured to carry out the instructions of a computer program. Processors and other such devices discussed herein are therefore configured to perform basic arithmetical, logical, and input/output operations.

Pump 100 and/or its components or subsystems discussed herein can include memory. Memory can comprise volatile or non-volatile memory as required by the coupled processor to not only provide space to execute the instructions or algorithms, but to provide the space to store the instructions themselves. In embodiments, volatile memory can include random access memory (RAM), dynamic random access memory (DRAM), or static random access memory (SRAM), for example. In embodiments, non-volatile memory can include read-only memory, flash memory, ferroelectric RAM, hard disk, floppy disk, magnetic tape, or optical disc storage, for example. The foregoing lists in no way limit the type of memory that can be used, as these embodiments are given only by way of example and are not intended to limit the scope of embodiments.

The components described herein can be constructed, programmed, configured, or otherwise adapted, to autonomously carry out a function or set of functions. Various components can comprise a real-world device, component, or arrangement of components implemented using hardware, such as by an application specific integrated circuit (ASIC) or field-programmable gate array (FPGA), for example, or as a combination of hardware and software, such as by a microprocessor system and a set of program instructions that implement the particular functionality, which (while being executed) transform the microprocessor system into a special-purpose device. Components also be implemented as a combination of the two, with certain functions facilitated by hardware alone, and other functions facilitated by a combination of hardware and software. Accordingly, each component can be realized in a variety of physically embodied configurations, and should generally not be limited to any particular implementation exemplified herein, unless such limitations are expressly called out. In addition, a component can itself be composed of more than one sub-component, each of which can be regarded as a component in its own right. Moreover, in the embodiments described herein, each of the various components corresponds to a defined autonomous functionality; however, it should be understood that in other contemplated embodiments, each functionality can be distributed to more than one component. Likewise, in other contemplated embodiments, multiple defined functionalities may be implemented by a single component that performs those multiple functions, possibly in parallel or series with, and/or complementary to other functions, or distributed differently among a set of components than specifically illustrated in the examples herein.

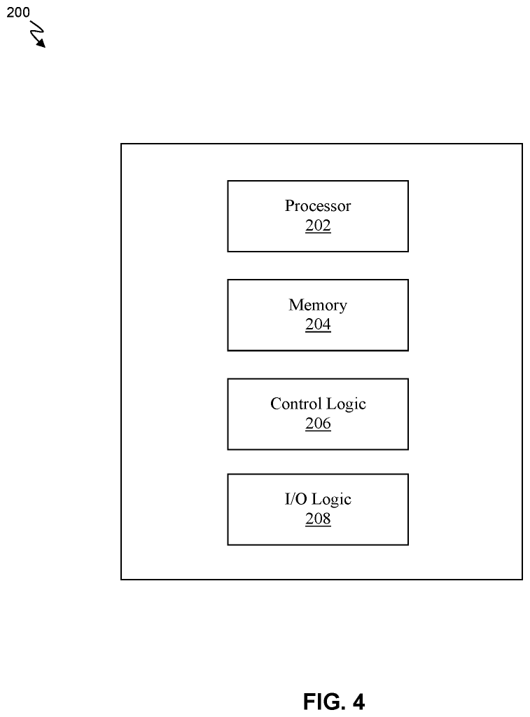

As discussed above, user interface 102 serves as a source of data input for pump 100 from a medical practitioner, pump programmer, or other user. User interface 102 can include a touchscreen display 104, keypad 106 or a combination of these or other user interface technologies. In embodiments, as will be described, user interface 102 can further include a controller subsystem (see FIG. 4).

Controller 108 is connected to the user interface and is responsible for ensuring that the pump 100 is controlled in the desired manner. Controller 108 is located in the housing 212 and controls operation of the motor 108 and drive components 108. In certain embodiments, controller 108 further controls operation of user interface 102. For example, the display controller subsystem can be implemented within controller 108, or be separate from controller 108 in other embodiments. Controller 108 can include one or more processors. Controller 108 may further include memory in some embodiments.

Motor 110 is operably coupled to controller 108 and pump components generally. Motor 110 is the primary means for directing drivetrain 112 (or drive components) to effect movement of a plunger head assembly. Drivetrain 112 can be a set of drive components that are at least partially located in the housing of the pump and which are responsible for mechanically directing infusion of fluid.

Pump 100 further includes either line power via a cord connected to power receptacle 114 or via a connector in a rack that connects to power receptacle 114. Battery 116 provides another alternate source of power to the infusion pump 100. Battery 116 can be fully enclosed in the housing, in embodiments.

Various electrical components and electrical circuitry 118 are located within the pump housing that are required for relaying or carrying out commands to controller 108 or within the system. Various outside devices may be connected to pump 100 as well through inputs, such as Ethernet connector 120 or USB port 122.

Speakers 124 are equipped to provide a full range of audio outputs including commands, alerts, and informative communications.

Plunger head sensors 126 and other sensors can be part of the system as well. Plunger head sensors 126, for example, can make various measurements for tasks such as characterizing syringes, detecting occlusions, and determining plunger position. Controller 108 utilizes information gained from sensors 126 and other components to assist in communications and decision-making. Although not specifically illustrated, it is to be appreciated and understood that in LVP pump embodiments, sensors 126 can comprise sensing devices for LVP applications.

Referring to FIG. 4, a block diagram for a controller subsystem for an infusion pump is depicted, according to an embodiment. Controller subsystem 200 can be implemented as part of controller 108 and/or user interface 102. Controller subsystem 200 generally comprises a processor 202, a memory 204, control logic 206, and I/O logic 208.

Processor 202 can include fixed function circuitry and/or programmable processing circuitry. Processor 202 can include any one or more of a microprocessor, a controller, a DSP, an ASIC, an FPGA, or equivalent discrete or analog logic circuitry. In some examples, processor 202 can include multiple components, such as any combination of one or more microprocessors, one or more controllers, one or more DSPs, one or more ASICs, or one or more FPGAs, as well as other discrete or integrated logic circuitry. The functions attributed to processor 202 herein may be embodied as software, firmware, hardware or any combination thereof. In embodiments, processor 202 comprises a programmable device configured for control, display, and user interface operations, as well as any other suitable operations, as will readily be appreciated by one of ordinary skill in the art.

Memory 204 can include computer-readable instructions that, when executed by processor 202 cause the pump 100 to perform various functions. Memory 204 can include can volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. In embodiments, memory 204 is operably coupled to processor 202 and can provide space to execute the instructions or algorithms of control logic 206 and/or I/O logic 208, and can also provide the space to store the instructions themselves, in certain embodiments.

Control logic 206 comprises software logic configured to operate an infusion pump. In an embodiment, programming logic is configured with various pump application program instructions (e.g., executable code, rules, and/or data) that control operation of the pump for a specific therapy or type of delivery (e.g., continuous delivery, intermittent delivery, pain control, chemotherapy, total parenteral nutrition, etc.). For example, a pump application program might contain instructions that define operation of a pump to accomplish various of the pump parameters. Pump application programs include, for example, pump protocols including both patient specific and non-patient specific pump parameters, and instructions for allocating memory, user interfaces, or algorithms for monitoring various sensors and driving a motor for the pump mechanism.

Control logic 206 can also be programmed or configured to access databases (often referred to as or including “drug libraries”) containing information relating to infusates that can be used with a specific pump, as well as information corresponding to dosing guidelines, drug concentrations, dose limits, and clinical advisories. Typically, a drug library contains a list of medications at predefined or standard concentrations, which in turn effectively determines safe dosing ranges.

In embodiments, control logic 206 can be configured to interface to an HIS to receive programming parameters related to operation of an infusion pump. In certain embodiments, the HIS, or corresponding pharmacy subsystem is configured to handle safety aspect checks before the protocol is sent to the pump.

I/O logic 208 comprises software logic configured to make logical decisions about what information to display to a user and what information to receive from a user. In embodiments, I/O logic 208 can display to a screen I/O, keypad I/O or other suitable input/output mechanism. In embodiments, I/O logic 208 can receive inputs from a keypad I/O and/or screen I/O or other suitable input/output mechanism. Further, I/O logic 208 can be integrated with controller 108 and the infusion algorithms for pump 100 via programming logic 206.

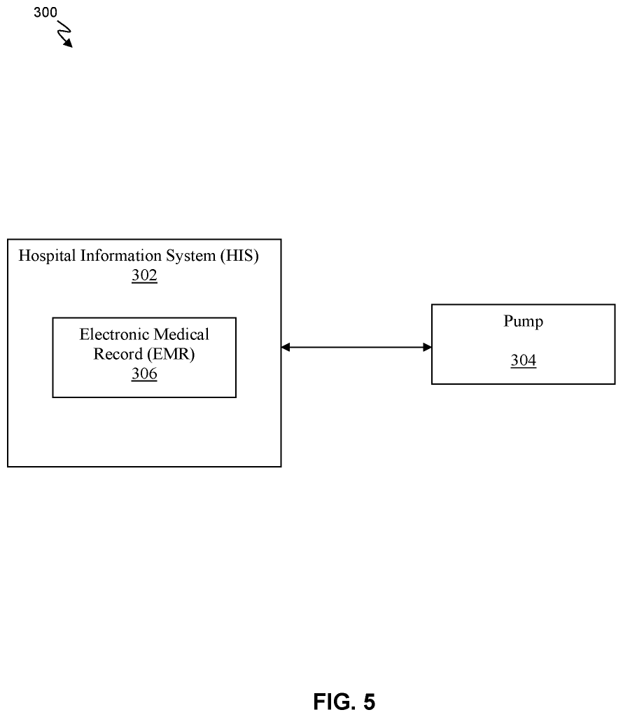

Referring to FIG. 5, an architecture block diagram for an infusion pump system is depicted, according to an embodiment. System 300 generally comprises a Hospital Information System (HIS) 302 in electronic communication with a pump 304. HIS 302 described in the figures and throughout this document, comprises the information or management system of a hospital, with all of its subcomponents and subsystems. HIS 302 includes a system providing healthcare related information that is integrated and is accessible by persons at a hospital or healthcare facility to assist in providing patient care. Such are comprehensive, integrated information systems designed to manage the medical, administrative, financial and legal aspects of a hospital and its service processing.

In an embodiment, HIS 302 can further include or manage electronic medical records (EMRs) 306 for patients. Such electronic records can include up-to-date medical histories, patient data, lab work, test results, prescriptions, imaging and diagnosis information for patients. HIS 302 can be configured to transmit data to a server for integration into the drug libraries in some embodiments. Likewise, data can be transmitted from a server to HIS 302 for informational, reporting, or patient care purposes.

In embodiments, HIS 302 and/or EMR 306 can transmit an infusion order to an infusion pump, such as pump 304, in an auto-programming (or “smart pump programming”) process. Auto-programming via an HIS is typically more efficient than manual programming entry by a user at a pump. In embodiments, HIS 302 and/or EMR 306 can ensure safety of programming parameters through medication safety software checks and correlated population from the ordering system where such parameters have been reviewed and approved prior to transmission to pump 304. Thus, auto-programming can help eliminate infusion errors and provide more prompt and timely patient care.

In an embodiment, an auto-programming process can be initiated at HIS 302 by a Bar Code Medication Administration configured to send an initial message that starts the pump process.

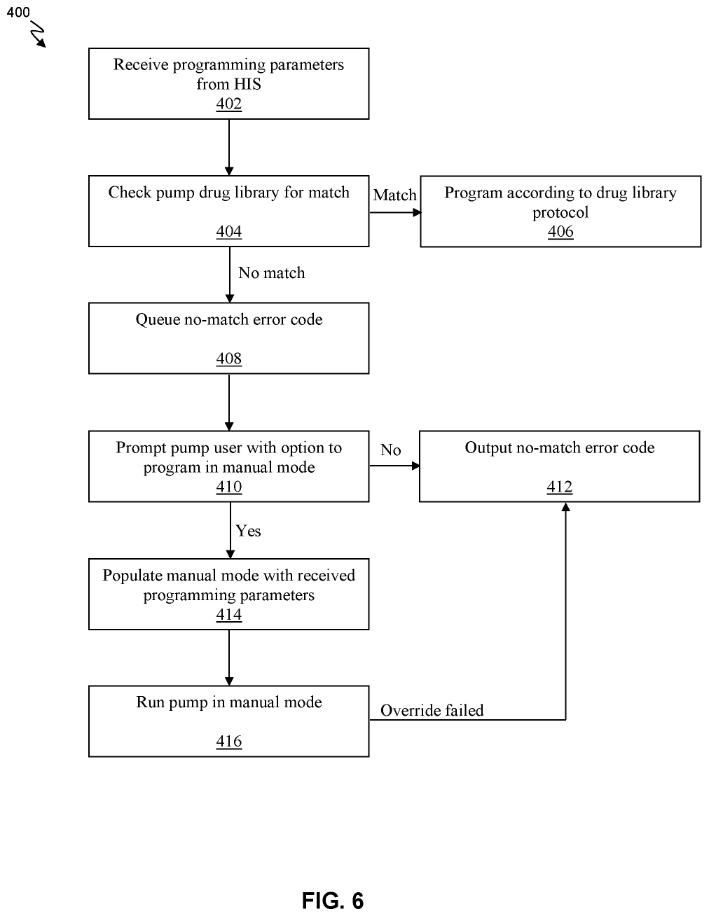

Referring to FIG. 6, a flowchart of a method 400 of programming an infusion pump is depicted, according to an embodiment. In an embodiment, method 400 can program one of the pumps described herein, such as pump 100 or pump 304. Further, the operations described as part of method 400 can be implemented in control logic 206 and/or I/O logic 208.

In an embodiment, method 400 generally comprises, at 402, receiving, by the infusion pump, programming parameters from an HIS. In an embodiment, the programming parameters are received as part of an autoprogramming or smart pump programming workflow. For example, the programming parameters can be entered by a physician as part of a computerized physician order entry, approved by a pharmacy, and transmitted over a network to the pump.

At 404, the pump checks its pump drug library for a protocol associated with the received programming parameters. In an embodiment, the pump can execute various levels of filtering to determine if the programming parameters have a corresponding drug library entry. For example, the pump can initially identify all drug library entries that match the programmed drug identifier or drug name, then the particular concentration(s) specified, then the delivery units specified, etc.

At 406, if the pump identifies a matching protocol, the pump can be programmed according to that particular drug library protocol in the typical autoprogramming workflow.

However, if the pump cannot identify a matching protocol at 404, instead of exiting the autoprogramming workflow and providing a no-match error code at the pump (and back to the HIS), the pump can queue the no-match error code at 408 and continue in the workflow.

At 410, the user is prompted with the option to program in manual mode with the received programming parameters. If the user declines the option to program in manual mode, the no-match error code, as well as any other appropriate error codes, are output at the pump, and back to the HIS, as appropriate at 412.

If the user accepts the option to program in manual mode, the pump is configured to run in a manual mode and the received programming parameters are populated as part of the manual mode operation at 414.

At 416, the pump is operated in manual mode. If the manual mode operation “override” fails, the no-match error code, as well as any other appropriate error codes, are output at the pump, and back to the HIS, as appropriate.

Optionally, and not shown in FIG. 6, data about the infusion can automatically flow back to the HIS for recording.

Accordingly, embodiments are configured such that the pump can maintain the autoprogramming workflow instead of exiting the workflow when no drug library match is found. Moreover, a new drug library entry does not need to be created or filled in from an empty drug library entry template.

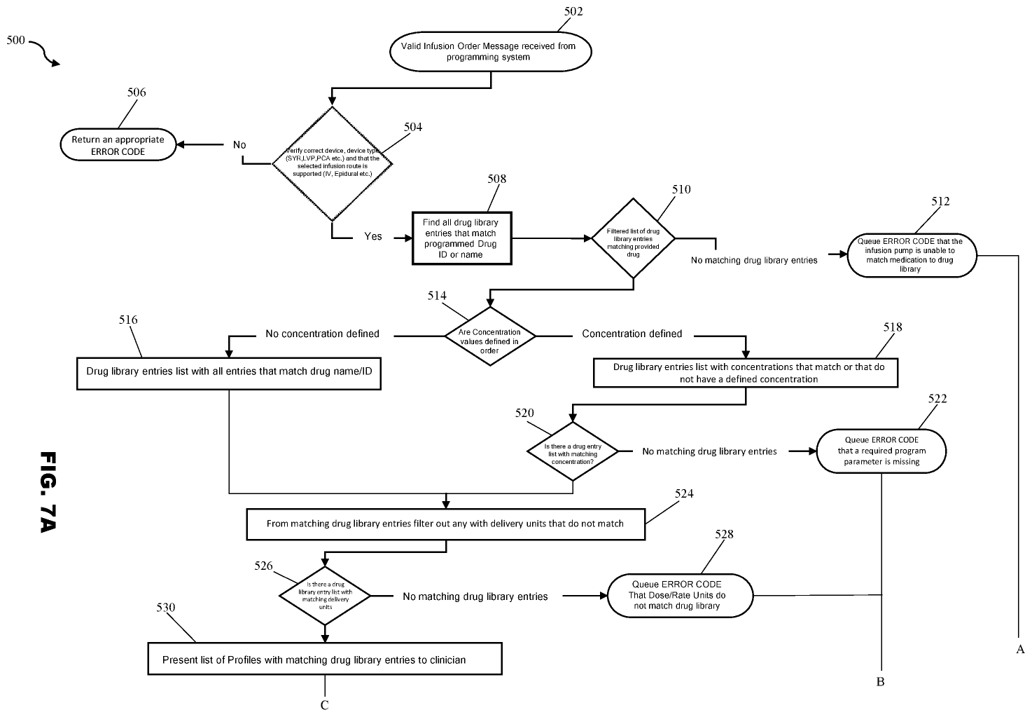

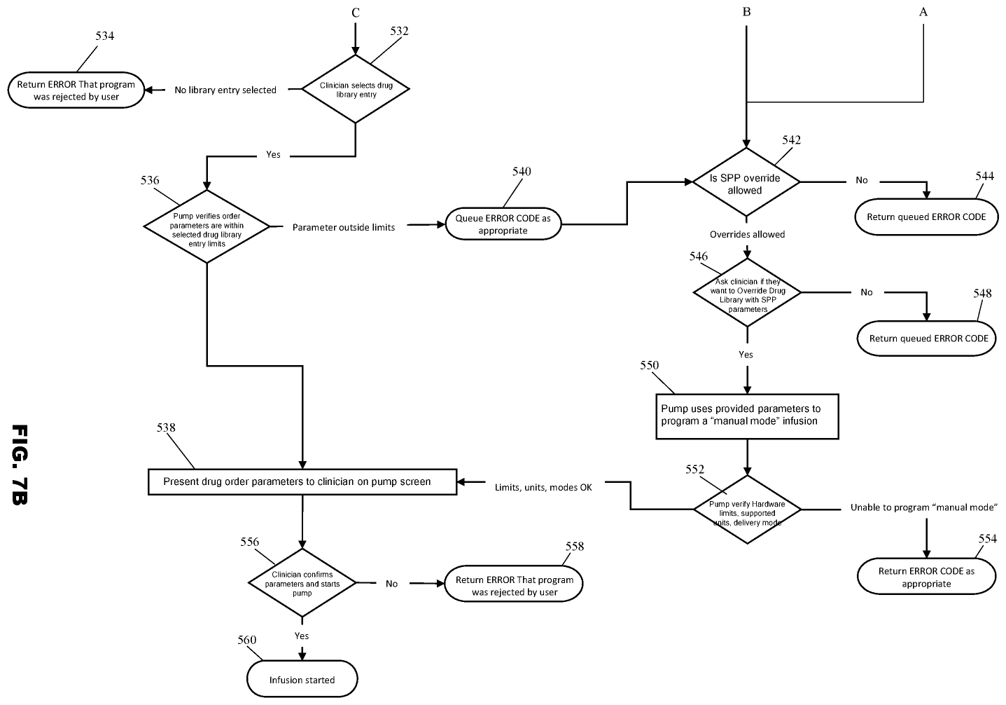

Referring to FIGS. 7A-7B, a flowchart of a method 500 of programming an infusion pump is depicted, according to an embodiment. Similar to FIG. 6, method 500 can program one of the pumps described herein, such as pump 100 or pump 304. Further, the operations described as part of method 500 can be implemented in control logic 206 and/or I/O logic 208.

At 502, a valid infusion order message is received by an infusion pump from a programming system, such as a CPOE on an HIS.

At 504, the pump verifies that the infusion order message includes the correct device, the correct device type (e.g. syringe, LVP, PCA, etc.), and that the selected infusion route is supported (e.g. IV, epidural, etc.).

At 506, if one of the device, device type, or infusion route does not match, an appropriate error code can be output. In embodiments, the error code can be output to the pump, as well as back to the operably coupled HIS.

At 508, if device, device type, and infusion route do match, the pump begins the process of filtering to find matching drug library entries. In an embodiment, the pump can find all drug library entries that match the received drug i.d. or drug name from the received infusion order message.

At 510, the pump provides a filtered list of drug library entries matching the drug provided in the received infusion order message.

At 512, if the pump does not find any matching drug library entries, an error code that the infusion pump is unable to match the received medication to its drug library is queued. Via path A, the received parameters are used to attempt a manual infusion (as will be described).

At 514, and continuing the filtering process, the pump determines if concentration values are defined in the received infusion order message.

At 516, if no concentration is defined in the received infusion order message, a list of drug library entries that match the drug name or drug i.d. is created.

At 518, if a concentration is defined in the received infusion order message, a list of drug library entries with concentrations that either match or do not have a defined concentration is created.

At 520, from a generated subset list, a check for a matching concentration is conducted. For example, the generated subset list can be checked for a matching concentration to the received concentration. If there are no matching drug library entries from 520, an error code that a required program parameter is missing is queued at 522. Via path B, the received parameters are used to attempt a manual infusion (as will be described).

At 524, from either 516 via the no-concentration-defined path, or 520 via the concentration-defined path, using the matched entries, the pump filters out any entries with delivery units that do not match the received parameters.

At 526, the pump determines if there is a drug library entry with matching delivery units. At 528, if there are no matching drug library entries, an error code that the dose or rate units do not match the drug library is queued at 528. Via path B, the received parameters are used to attempt a manual infusion (as will be described).

At 530, if the delivery units match, a list of profiles with matching drug library entries is presented to the clinician.

Continuing via path C in FIG. 7B, the clinician selects a drug library entry. At 534, if the clinician does not select a library entry, an appropriate error code can be output. For example the error code can indicate that programming was rejected by the user. In embodiments, the error code can be output to the pump, as well as back to the operably coupled HIS.

At 536, if the clinician selects a drug library entry, the pump verifies the order parameters are within the selected drug library entry limits.

At 538, the drug order parameters are presented to the clinician on the pump user interface.

Returning to 536, and if the pump determines that an order parameter is outside of the selected drug library entry limits, an appropriate error code is queued at 540.

In contrast to traditional methods in which the workflow is exited and errors are reported, errors 512, 522, 528, and 540 are queued instead.

Returning to paths A and B, at 542, the pump determines whether an autoprogramming override is allowed. In an embodiment, a pharmacist user can implement whether or not autoprogramming overrides are allowed by setting an option in the pump's drug library. For example, this can be done prior to any autoprogramming, such as an initial configuration of the drug library. Subsequently, at 542, the pump checks the “autoprogramming override allowed” option to determine if the pump can override.

At 544, if an autoprogramming override is not allowed, the autoprogramming workflow is exited and the appropriate queued error code is returned. In embodiments, the error code can be output to the pump, as well as back to the operably coupled HIS.

At 546, if an autoprogramming override is allowed, control logic prompts the user (e.g. the clinician) to override the drug library with the received parameters. If the user chooses not to override the drug library with the received parameters, the autoprogramming workflow is exited and the appropriate queued error code is returned at 548. In embodiments, the error code can be output to the pump, as well as back to the operably coupled HIS.

At 550, if the user chooses to override the drug library with the received parameters, the pump is populated with parameters received from the HIS to program a “manual mode” infusion.

At 552, the pump verifies the hardware limits, supported units, and delivery mode. If, from 552, the pump is unable to program in manual mode, the autoprogramming workflow is exited and the appropriate queued error code is returned at 554.

However, if the hardware limits, supported units, and delivery mode are acceptable, the drug order parameters are presented to the clinician on the pump user interface at 538 (as in the standard autoprogramming workflow). In another embodiment, the minimum values needed to run are a drug name and a rate.

At 556, the clinician confirms the parameters and starts the pump. At 558, if the clinician rejects the parameters, the autoprogramming workflow is exited and the appropriate queued error code is returned.

At 560, the infusion is started. In an embodiment, the infusion started message (with appropriate accompanying data about the infusion) can be sent back to the HIS. The HIS can flag the infusion if it is different than what was originally commanded. A clinician operating the HIS can accept the infusion from there and the HIS can be subsequently updated.

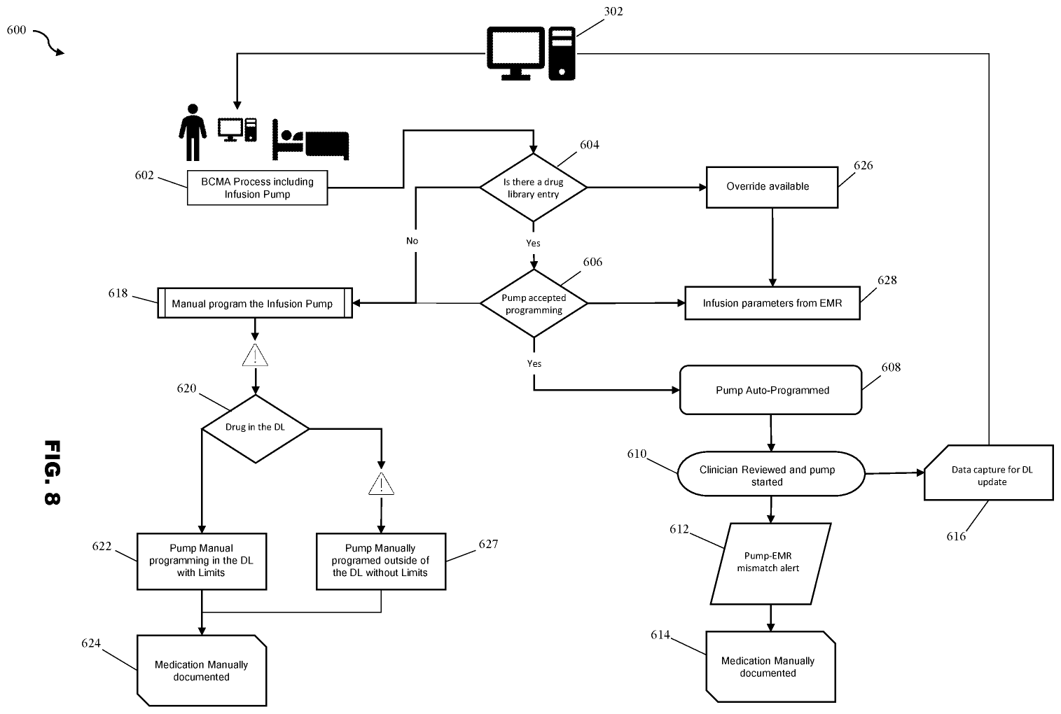

Referring to FIG. 8, a flowchart of a method 600 of intelligent interoperability between an HIS and an infusion pump is depicted, according to an embodiment. In embodiments, existing systems and methods of interoperability are improved by the systems and methods of programming an infusion pump described herein.

For example, HIS 302 and pump 304 (not shown) can be utilized in method 600. At 602, HIS 302 can command or otherwise instruct an infusion for pump 304. A BCMA sub-process can check that an infusate container is labeled with the appropriate barcode corresponding to a particular patient and an applicable order for the patient. At 604, method 600 determines if there is a drug library entry in pump 304. From 604, method 600 can enter three primary sub-processes.

In a first primary sub-process from 604, method 600 determines that an appropriate drug library entry for the instructed infusion is present in pump 304. At 606, pump 304 accepts the programming of the instructed infusion. At 608, pump 304 is auto-programmed.

At 610, a clinician can review the programming of 608 and start pump 304. At 612, if method 600 determines a pump-EMR mismatch, an alert is presented. At 614, the medication is auto-documented, for example, on the pump and/or on EMR 306. Returning to 610, method 600 can execute a data capture for a drug library update at 616. In an embodiment, at part of 616, method 600 can document one of the four categories of compliance for the infusion: (1) started non-drug library infusions, (2) started drug library infusions, (3) SPP drug library infusions, and (4) SPP EMR non-drug library infusions. Such documentation can be transmitted back to HIS 302.

In a second primary sub-process from 604, method 600 determines there is no appropriate drug library entry for the instructed infusion and no override is available, and proceeds to 618 for manual programming of pump 304. At 620, method 600 determines if the drug of the instructed infusion is in the drug library. At 622, if the drug is found in the drug library, pump 304 is manually programmed using the limits defined by the drug library. At 624, the medication is manually documented by a clinician. In embodiments, the documentation can optionally be used to back-associate the drug in the drug library. Alternatively, returning to 620, if the drug is not found in the drug library, pump 304 can be programmed outside of the drug library without limits at 627. Again, at 624, the medication can be manually documented by a clinician.

In a third primary sub-process from 604, method 600 determines there is no appropriate drug library entry for the instructed infusion, but an override is available at 626. In an embodiment, pump 304 is programmed according to method 400 and/or method 500 or subcomponents of embodiments of the methods using infusion parameters from EMR 306 at 628. Method 600 can then proceed as described with respect to the first primary sub-process at 606.

Various embodiments of systems, devices, and methods have been described herein. These embodiments are given only by way of example and are not intended to limit the scope of the claimed embodiments. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed embodiments.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. An infusion pump including a processor, memory operably coupled to the processor, and control logic comprising instructions that, when executed, cause the processor to:
receive programming parameters corresponding to an infusion order from a Hospital Information System (HIS), the programming parameters having been verified by the HIS;
determine if the received programming parameters match a drug library protocol in a drug library of the infusion pump;
when the programming parameters do not match any drug library protocol in the drug library, queue a no-match error code;
populate a manual mode with the received programming parameters; and
operate the infusion pump in the manual mode using the populated programming parameters.

2. The infusion pump of claim 1, wherein the control logic comprises instructions that, when executed, cause the processor to further:
output the queued no-match error code to the HIS when the manual mode infusion fails.

3. The infusion pump of claim 1, wherein the control logic comprises instructions that, when executed, cause the processor to further:
prompt a user with the manual mode operation option; and
receive an acceptance of the manual mode operation option prior to populating the received programming parameters.

4. The infusion pump of claim 3, wherein the control logic comprises instructions that, when executed, cause the processor to further:
determine that an autoprogramming override is allowed prior to prompting the user with the manual mode operation option.

5. The infusion pump of claim 1, wherein the control logic comprises instructions that, when executed, cause the processor to further:
wherein populating the manual mode with the received programming parameters includes entering the received programming parameters without creating a populated or partially populated drug library entry.

6. The infusion pump of claim 1, wherein the control logic comprises instructions that, when executed, cause the processor to further:
filter through the drug library of the infusion pump, wherein the no-match error code is based on the filtering and is at least one of a drug not entered in the drug library, a required programming parameter missing, or a dose unit or rate unit does not match the drug library.

7. The infusion pump of claim 1, wherein the control logic comprises instructions that, when executed, cause the processor to further:
verify a hardware limit, a supported unit, and a delivery mode of the manual mode are compatible with the infusion pump prior to operating the infusion pump in the manual mode.

8. The infusion pump of claim 1, wherein the control logic comprises instructions that, when executed, cause the processor to further:
transmit manual mode information to the HIS.

9. A method of programming an infusion pump, the method comprising:
receiving programming parameters corresponding to an infusion order from a Hospital Information System (HIS), the programming parameters having been verified by the HIS;
determining if the received programming parameters match a drug library protocol in a drug library of the infusion pump;
when the programming parameters do not match any drug library protocol in the drug library, queuing a no-match error code;
populating a manual mode with the received programming parameters; and
operating the infusion pump in the manual mode using the populated programming parameters.

10. The method of programming an infusion pump of claim 9, further comprising:
outputting the queued no-match error code to the HIS when the manual mode infusion fails.

11. The method of programming an infusion pump of claim 9, further comprising:
prompting a user with the manual mode operation option; and
receiving an acceptance of the manual mode operation option prior to populating the received programming parameters.

12. The method of programming an infusion pump of claim 11, further comprising:
determining that an autoprogramming override is allowed prior to prompting the user with the manual mode operation option.

13. The method of programming an infusion pump of claim 9, wherein populating the manual mode with the received programming parameters includes entering the received programming parameters without creating a populated or partially populated drug library entry.

14. The method of programming an infusion pump of claim 9, further comprising:
filtering through the drug library of the infusion pump, wherein the no-match error code is based on the filtering and is at least one of a drug not entered in the drug library, a required programming parameter missing, or a dose unit or rate unit does not match the drug library.

15. The method of programming an infusion pump of claim 9, further comprising:
verifying a hardware limit, a supported unit, and a delivery mode of the manual mode are compatible with the infusion pump prior to operating the infusion pump in the manual mode.

16. The method of programming an infusion pump of claim 9, further comprising:
transmitting manual mode information to the HIS.

17. An infusion pump comprising:
means for receiving programming parameters corresponding to an infusion order from a Hospital Information System (HIS), the programming parameters having been verified by the HIS;
means for determining if the received programming parameters match a drug library protocol in a drug library of the infusion pump;
when the programming parameters do not match any drug library protocol in the drug library, means for queuing a no-match error code;
means for populating a manual mode with the received programming parameters; and
means for operating the infusion pump in the manual mode using the populated programming parameters.

18. The infusion pump of claim 17, further comprising:
means for outputting the queued no-match error code to the HIS when the manual mode infusion fails.
